(19) United States
(12) Patent Application Publication
(10) Pub. No.: US 2022/0118294 A1
(43) Pub. Date: Apr. 21, 2022

(54) SEE-THROUGH FACE MASK AND FILTRATION SYSTEM FOR THE SAME

(71) Applicant: The Trustees of Dartmouth College, Hanover, NH (US)

(72) Inventors: Zi Chen, Norwich, VT (US); Jing Fan, New York, NY (US); Matthew Liao, Boston, MA (US)

(21) Appl. No.: 17/503,166

(22) Filed: Oct. 15, 2021

Related U.S. Application Data
(60) Provisional application No. 63/092,301, filed on Oct. 15, 2020.

(51) Int. Cl. A62B 23/02; A62B 18/08; A62B 18/02; A61F 9/02; A41D 13/11; B01D 45/06; B01D 45/08; B01D 50/20

(52) U.S. Cl. CPC A62B 23/02; A62B 18/084; A62B 18/086; A62B 18/08; B01D 50/20; A61F 9/029; A41D 13/11; B01D 45/06; B01D 45/08; A62B 18/02

(57) ABSTRACT

This invention provides a see-through full face mask and hood that integrates effective filtration for both inhaled and exhaled air. The mask includes a transparent visor mounted on a headband and surrounded by a frame. The headband or the frame (sides, bottom, etc.) can include a filtration assembly that allows passive inflow and outflow of air in a manner that is highly breathable. The frame generally seals against the users face in a comfortable manner. The visor can be surrounded by a subframe that can be moved upwardly out of engagement with a base frame—which remains attached to the wearer to expose the user's mouth for eating. The filtration assembly defines a plurality of tortuous or circuitous-path filtration tubes that employ bends, baffles, charged surfaces and/or other internal barriers to redirect airflow and thereby trap microdroplets containing pathogens until such pathogens can be rendered inactive.

### FIELD OF THE INVENTION

This invention relates to personal protective equipment (PPE) for use in prevention of exposure to bacterial and viral infectious diseases and other airborne toxins, and more particularly to face masks that filter breathable air.

### BACKGROUND OF THE INVENTION

Human coronavirus (referred to herein as “SARS-CoV-2”), has emerged as a significant threat to world health, and is responsible for the current pandemic of 2020 in which it is referred to as COVID-19. Like many contagious virus strains, SARS-CoV-2 is released from an infected person as an aerosol during breathing, coughing, sneezing, blowing of his or her nose, or just touching the face. Such aerosolized viruses thereby spread through the air, remaining in air for hours. Such viruses may also adhere to various surfaces, including those that make up PPE components, including but not limited to, masks, gloves, gowns, hazmat suits, etc. As the virus can remain active for some time (hours or even days) on surfaces, they pose a continuing risk to non-infected persons, including patients, health care workers and bystanders. Those active viruses can infect non-infected persons through inhalation of the aerosolized form, or through hand/finger contact with viruses on a surface, and subsequent transfer to a mucous membrane, mouth or other orifice via the hand/finger.

PPE is essential to slow the spread of airborne pathogens, like COVID-19. However traditional face-blocking PPE has many disadvantages. For example, face shields can be desirable in certain settings since they block direct projection and receipt of airborne droplets, while providing good visibility for the wearer and ample airflow. However, the openness of the face shield also allows for indirect passage of a large volume of aerosolized pathogens in the form of microdroplets. Traditional face masks, which are placed over the nose and mouth perform better in filtering out microdroplets, but obstruct facial expressions, muffle speech and can be uncomfortable for long-term wearing. Additionally, masks must be replaced or cleaned at frequent intervals to maintain their effectiveness and avoid contamination from the wearer's own bacterial/biofilm buildup. Additionally, the use of disposable masks is becoming a problem in terms of waste management and proper disposable of an item that can be considered biohazardous.

It is desirable to provide a PPE for covering the facial region of a user that allows for comfort, visibility, exposure of facial features, long-term wearability and good filtration of both inhaled and exhaled ambient air.

### SUMMARY OF THE INVENTION

This invention overcomes disadvantages of the prior art by providing a see-through full face mask, half face mask and hood that integrates effective filtration for both inhaled and exhaled air. The mask includes a transparent visor mounted on a headband or straps, and surrounded by a frame. The headband, half mask top, or the frame (sides, bottom, etc.) can include a filtration assembly that allows passive inflow and outflow of air in a manner that is highly breathable. The frame generally seals against the users face in a comfortable manner. The visor can be surrounded by a subframe that can be moved upwardly out of engagement with a base frame—which remains attached to the wearer to expose the user's mouth for eating. Additionally or alternatively, the front of the visor can include a drinking plug that can be removed to allow a straw to be inserted and reach the wearer's mouth for drinking. In various embodiments, the filtration assembly can define multiple layers of cloth and/or filter material. Illustratively, the filtration assembly defines a plurality of tortuous or circuitous-path filtration tubes that employ bends, baffles, or other internal barriers to redirect airflow, and thereby trap, microdroplets containing pathogens until such pathogens can be rendered inactive. The tubes can be formed from a strain-inducing stretching process, molding or printing a series of baffles with misaligned openings, creating a complex matrix of internal walls and offset stem tubes and diverting domes, or providing a long (e.g. helical), electrically charged tube that attracts microdroplets to its walls. The filtration assemblies can include automatic or manual adjustment for air temperature. Additional sensors, such as a temperature sensor, cough detector, etc. can be provided, and can either store or transmit appropriate telemetry to the user or another interested party.

In an illustrative embodiment, a see-through face mask is provided. The face mask includes a frame having at least one of a top, bottom and sides, which engages a face of a wearer and provides a cushioned approximate seal thereagainst. A strap system maintains the frame against the wearer's face. A visor that is substantially transparent is supported by the frame, and a filtration assembly resides in the frame, and allows passage of inhaled and exhaled air therethrough. Illustratively, the filtration assembly comprises a plurality of tortuous filtration tubes that are constructed and arranged to trap microdroplets in the air as it passes therethrough. The tortuous filtration tubes can each comprise a strain-engineered elastomer structure. The tortuous filtration tubes can further comprise a plurality of internal baffles with openings in an offset arrangement therebetween. The internal baffles can include stem tubes and diverting domes in fluid communication with the stem tubes. The frame and the visor can define a full face mask, and the filtration assembly is located in at least one of the top and the bottom of the frame. Alternatively, the frame and the visor can define a half face mask, and the filtration assembly is located in at least one of the top and the bottom of the frame.

In another illustrative embodiment, a filtration assembly for a face mask is provided, and can comprise a plurality of tortuous filtration tubes that are constructed and arranged to trap microdroplets in the air as it passes therethrough. Illustratively the tortuous filtration tubes can each comprise a strain-engineered elastomer structure. Alternatively, the tortuous filtration tubes can comprise a plurality of internal baffles with openings in an offset arrangement therebetween. The internal baffles can include stem tubes and diverting domes in fluid communication with the stem tubes. The face mask can comprise either a full face mask or a half face mask. Additionally, the filtration assembly can be located in at least one of a top and a bottom of a frame of the face mask.

### BRIEF DESCRIPTION OF THE DRAWINGS

The invention description below refers to the accompanying drawings, of which:

FIG. 1 is a side view of a see-through full face mask with a fabric filtration assembly according to a general embodiment;

FIGS. 1A and 1B are front and rear perspective views of an embodiment of a see-through full face mask with fabric filtration assembly according to an exemplary embodiment;

FIGS. 2 and 3 are front and side views, respectively of a see-through full face mask employing filtration tubes in the form of strain-engineered, polymer (elastomeric) tortuous filtration tubes in the bottom hosting compartment of the mask according to an embodiment;

FIGS. 4-6 are more detailed views of the filtration tubes of FIGS. 2 and 3;

FIG. 7 is a perspective view of a see-through full face mask according to an embodiment, having a filtration tube assembly integrated in the headband;

FIG. 8 is a more detailed fragmentary view of a visor retaining clip in the frame of the mask of FIG. 7

FIG. 9 is a further perspective view of the mask of FIG. 7;

FIG. 10 is an exposed perspective view of a segment of a filtration tube having a series of baffles with wedge-shaped offset openings therebetween to trap microdroplets;

FIG. 11 is an exposed perspective view of the segment of the filtration tube of FIG. 10 showing a path of redirected airflow through the baffle and offset opening arrangement;

FIG. 12 is an exposed perspective view of the segment of the filtration tube of FIG. 10 showing the deposition of microdroplets on the baffle surfaces based upon path of redirected airflow through the baffle and offset opening arrangement;

FIG. 13 is a side view of a helical filtration tube that is electrically charged to attract microdroplets passing therethrough;

FIG. 14 is a fragmentary perspective view of a segment of the interior of the filtration tube of FIG. 13, showing a positively charged half and a negatively charged half;

FIG. 15 is a side view of the positively charged half of FIG. 14 showing attraction of negatively charged and neutrally charged (polarized) microdroplets therethrough;

FIG. 16 is side view of the negatively charged half of FIG. 14 showing attraction of positively charged and neutrally charged (polarized) microdroplets therethrough;

FIG. 17 is a side cross section of a segment of a filtration tube having a complex structure of baffles, stem tubes and domes for redirecting airflow and trapping microdroplets;

FIG. 18 is side view of the filtration tube of FIG. 17 shown fully enclosed;

FIGS. 19 and 20 are perspective views of a see-through full face mask having a movable visor to allow access to the wearer's mouth for eating and other activities according to an embodiment in a closed and an open orientation, respectively;

FIGS. 21 and 22 are perspective views of the mask of FIGS. 19 and 20 showing a drinking plug in an attached and detached state so as to allow insertion of an exemplary drinking straw, respectively;

FIG. 24 is a more detailed perspective view of the drinking plug of FIGS. 21 and 22;

FIG. 25 is a rear-oriented perspective view of a see-through full face mask with a drinking plug according to another embodiment;

FIG. 26 is a perspective view of a see-through full face hood with a square cross section and flexible fabric bottom, according to an exemplary embodiment;

FIG. 27 is a perspective view of a see-through full face hood with a circular or elliptical cross section and zippered side closure, according to an exemplary embodiment;

FIG. 28 is a frontal perspective view of a see-through half face mask incorporating a filtration assembly according to the various embodiments herein;

FIG. 29 is a side perspective view of the half face mask of FIG. 28;

FIGS. 30 and 31 are front and side perspective views of a see-through full face mask according to an embodiment, having an antiviral or particulate filter integrated at the base of the mask, respectively;

FIGS. 32 and 33 are perspective views of a see-through half face mask having a movable visor to allow access to the wearer's mouth for eating and other activities according to an embodiment in a closed and an open orientation, respectively;

FIGS. 34 and 35 are perspective views of the mask of FIGS. 32 and 33 showing a drinking plug in an attached and detached state so as to allow insertion of an exemplary drinking straw, respectively;

FIG. 36 is a perspective view of a see-through half face mask according to an embodiment, having a polypropylene filter integrated at the base of the mask and sound-amplifying aluminum sheet pieces integrated at the top of the mask;

FIGS. 37 and 38 are top and bottom perspective views of the mask of FIG. 36, respectively;

FIG. 39 is a perspective view of a see-through half face mask frame according to an embodiment, having an open clamp mechanism to carry a fresh polypropylene filter integrated at the base of the mask;

FIGS. 40 and 41 are front-oriented and rear-oriented perspective views of the mask of FIG. 39, respectively;

FIG. 42 is a further perspective view of the mask of FIG. 39, shown applied on a wearer's head;

FIG. 43 is a side view of a wearable see-through half face mask according to an embodiment, having an open clamp mechanism to carry a fresh polypropylene filter integrated at the base of the mask, a drinking plug in an attached state, and accordion-shaped flexible ridges at the top of the mask;

FIGS. 44, 45, and 46 are front-oriented, rear-oriented, and bottom-oriented perspective views of the mask of FIG. 36, respectively, and;

FIGS. 47 and 48 are perspective views of the mask of FIGS. 43-46 showing the clamp mechanism in an attached or detached state so as to allow insertion of a polypropylene filter, respectively.

### DETAILED DESCRIPTION

### I. GENERAL PROPERTIES OF SEE-THROUGH FULL-FACE MASK

The following description relates to various exemplary embodiments of a see-through full face mask. The mask is generally characterized by a clear/transparent polymer front panel or visor that surrounds the entire frontal face in the manner of a face shield, and includes retaining straps at the top and bottom thereof that respectively surround the head and neck to secure the panel with respect to the face at a spacing therefrom that provides a substantial, enclosed airspace. This enhances comfort by avoiding close contact between the panel and the wearer's face. The visor is sealed with respect to the face by a sealing/securing structure that engages the face at the sides top and bottom. At least a portion of the sealing/securing structure can include a filtration assembly that blocks passage of unfiltered air into or out of the airspace. The filtration assembly can include a novel construction of passages that is described further below.

Basic Embodiment.

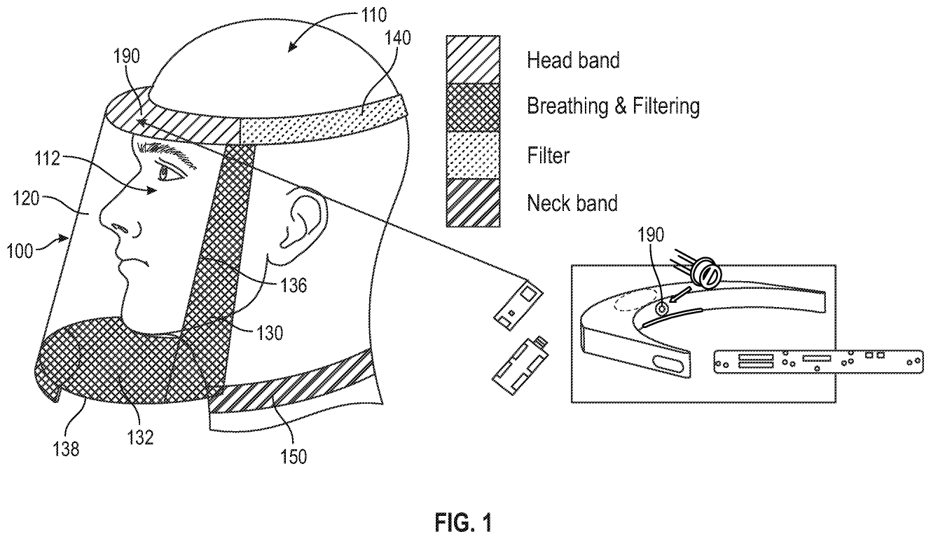

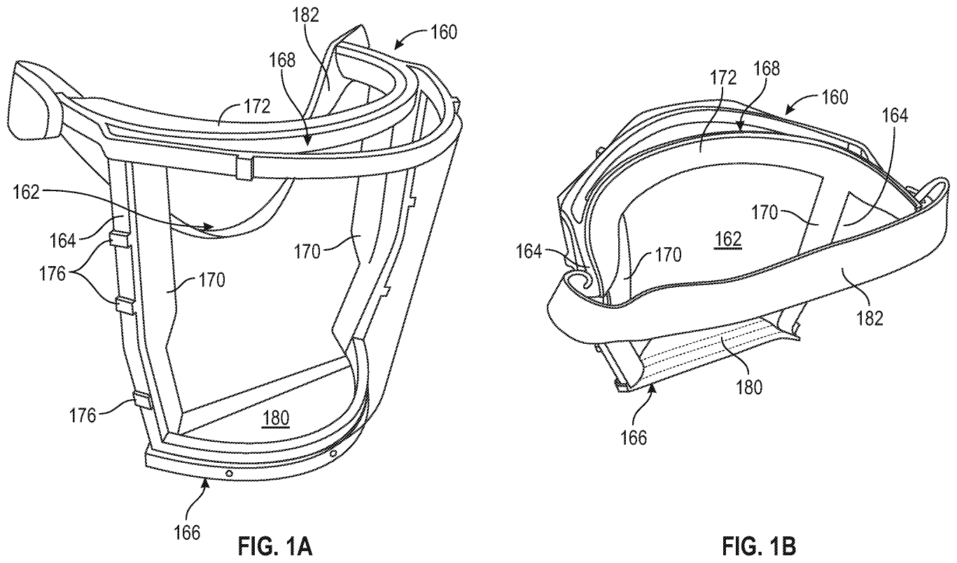

FIG. 1 depicts a basic embodiment for the see-through full face mask 100 according to the invention, shown applied to a wearer's head 110 and face 112. The visor 120 is transparent and can be constructed from a lightweight, biocompatible polymer material, such as anti-fog polycarbonate (PC) and polydimethylsiloxane (PDMS) of appropriate thickness. The visor (e.g. rigid/semi-rigid) 120 is secured using a wrap-around headband 140 that can be constructed similarly is similar to those of existing face shields with a good seal. Three (e.g.) cotton cloth pockets (130) are located on the two sides (left side being visible) and the bottom (132) of the visor to engage the wearer's face 112, respectively, to form a 3D structure for easy, optional insertion of middle-layer filters on each side. Moreover, soft rubber bands can be integrated into the cotton cloth the bias it against the face, so as to provide a good seal. Cotton cloth 130, 132 and a headband and/or the neckband 150 can be sewed together. For the PC visor embodiment, cotton cloth can be affixed to the PC film with an appropriate (e.g.) vinyl adhesive. The cotton cloth sides and bottom 130, 132 in this embodiment provide the breathing filter material. Because this region defines a significantly larger filter area than regular face masks, the breathing resistance of this mask 100 is typically significantly lower than directly worn face masks constructed from a similar type, density and/or thickness of filter material (as breathing resistance is inversely proportional to the filter area). In an embodiment the sides and bottom 130, 132 can be constructed from a double-layer cotton pocket with the option of adding a middle-layer melt-blown polypropylene filter (also employed as commercial N95 mask filter material). This additional material can further enhance filtration of breathable air.

Design and fabrication of see-through full face mask (100) with deformable visor 120 for this, and other, embodiments herein can include providing PDMS films with desired thickness can include squeezing/extruding PDMS liquid mixture between two rigid, molds/plates followed by high-temperature curing. Such construction techniques should be clear to those of skill. The resultant molded PDMS film can be coated with a thin polyvinyl alcohol (PVA) layer to eliminate fogging, in a manner clear to those of skill. Since the PDMS film is relatively flexible, as is the cotton (or other fabric) cloth, a rigid wire 136, 138 can be adhered, sewed (or otherwise secured) into the cotton cloth 130, 132 at the edge connecting to PDMS for structure support (bold black edge in FIG. 1). In an alternate embodiment a 3D printer can be used to provide a thin resin wire for this purpose.

Notably, the face mask 100 can be cleaned by removing the middle-layer filter (if any), and washing with disinfecting soap. Once dry it is ready for reuse. The middle-layer filter can additionally, or alternatively, be disinfected with UVC, and reused if desired. In further embodiments, the middle layer (or other filtration surfaced described herein) can include additional, potentially impregnated antibacterial, antiviral and/or absorbent) compounds, such as activated charcoal, nanoargentic silver, nanoparticulate copper, etc.

Design and fabrication of see-through full face mask according to another embodiment can include the use of tortuous respiratory tubes in addition to, or in alternative to, a cloth structure with middle-layer filter media. Traditional face masks require the use of a significant quantity of filter materials, which are often expensive and in short supply, and yet the masks made are uncomfortable for long-time wear. The design a new face mask that minimizes the use of filter materials and with significantly enhanced comfort for long-hour usage. By way of useful background, reference is made to Chen, Z., Majidi, C., Srolovitz, D. J., and Haataja, M. P. (2011) Tunable Helical Ribbons, Applied Physics Letters, 98, 011906; Guo, Q., Anil K. Mehta, Martha A. Grover, Wenzhe Chen, David G. Lynn, Chen, Z. (2014) Shape Selection and Instability in Helical Ribbons, Applied Physics Letters, 104, 211901; and Yu, X., Zhang, L., Hu, Hannah Grover, Shicheng Huang, Dong Wang, and Chen, Z. (2017). Shape Formation of Helical Ribbons Induced by Material Anisotropy, Applied Physics Letters, 110: 091901, the teachings of each of which are incorporated by reference. These references relate generally to construction of three-dimensional helical ribbon shapes from 2D precursors. This technique has since been widely adopted to fabricate spontaneous three-dimensional structures, including twisted structures that resemble looping guts in developing chick embryos. It is contemplated that a face mask with a filtration assembly incorporating very long, tortuous respiratory tubes to can be used to optimize comfort (of breathing) and safety. It is further contemplated that respiratory tubes are sufficiently long and twisted that there is a really high chance the viral droplets or aerosols will hit the inner wall sooner or later and be trapped locally or at least be significantly slowed down on its way to the inner space of the face mask. As long as the time it takes for the viral droplets or aerosols to travel from the outlet to the inlet exceeds the time SARS-CoV-2 remains viable, then the new face mask is very safe to use even without the presence of expensive filters that are often in short supply and meanwhile the face mask provides better comfort because the air can still go in and out almost freely.

FIGS. 1A-1B depict opposing perspective views of an embodiment of a rigid/semi-rigid visor see-through full face mask 160 similar in structure and function to the above-described face mask 100. This version surrounds the visor 162 with a polymer frame 164 that extends in a somewhat trapezoidal geometry from a narrower bottom 166 to a wider, top-mounted head band 168. The vertically oriented sides of the frame 166 are oriented so as to conform to the general dimensions of an average face of a given size. The frame sides 166 and head band can, respectively, include strips 170 and 172 of a compliant material, such a silicone and/or foam, that conforms to the face to provide a relatively airtight seal between the mask and wearer's face, and also provides enhanced comfort to the wearer. A set of clips (or other appropriate structures) 176 secures the visor 162 to the frame 164.

The mask 162 bottom 166 includes a filtration assembly 180 at the bottom 166, which is sized and arranged to comply against the wearer's chin in a manner clear to those of skill. Additional sealing structures, such as a silicone and/or foam edge can be provided in various embodiments. The filtration assembly can be constructed from one or more layers of cloth (with or without/free-of the above-described middle layer); or can be constructed using tortuous filtration tubes as described below.

The mask 160 of the above embodiment can be secured to the wearer's face by one or more straps, including a fixed or adjustable head strap 18 and an opposing neck strap (not shown). The neck strap (and optionally the head strap 182) can include a fastening mechanism and appropriate padding for comfort—for example a hook-and-loop or buckle-based fastening arrangement that allows for size adjustment at an appropriate location along the length of the strap.

### II. TORTUOUS OR CIRCUITOUS FILTRATION TUBES

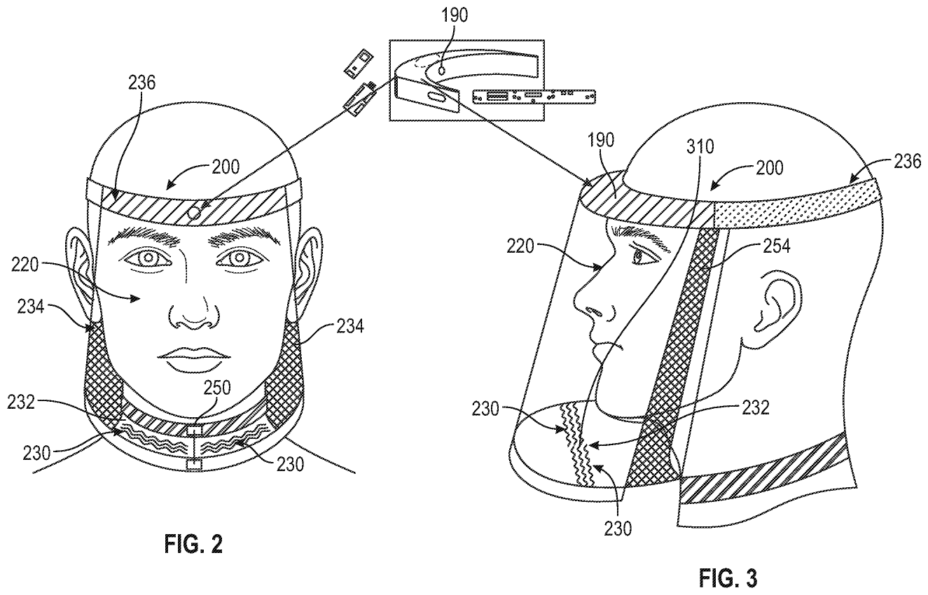

Reference is made to FIGS. 2 and 3, in which the filtration assembly 230 of the face mask 200, with transparent, deformable, visor 220, can be analogized with the twisted guts of chicken embryo, as such, are formed due to the differential growth between the gut tube and mesentery. In this embodiment, the twisted or tortuous (also termed “circuitous-path”) filtration assembly 230 is located in the bottom (e.g.) fabric panel 232 of the mask 200, termed a “hosting compartment”. As described below, in various embodiments, the twisted filtration assembly can be built into the (e.g. flexible fabric) sides 234 and/or top 236 of the mask 200.

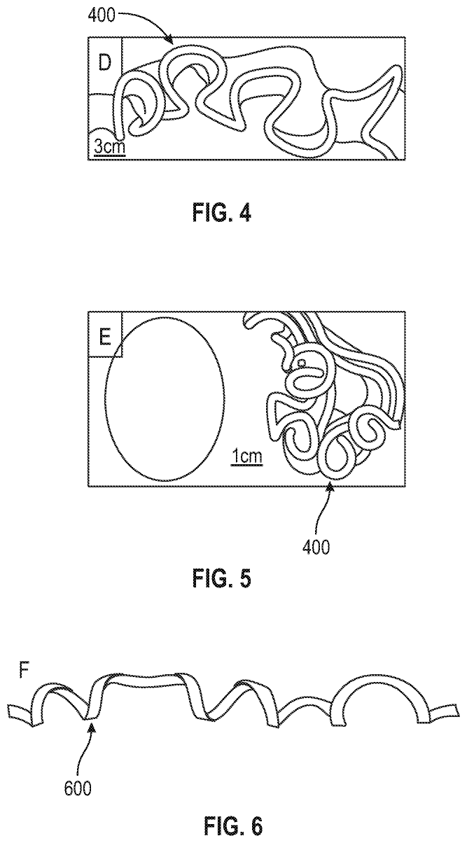

This twisted relationship of the filter tubes provides a ratio in which the total length of the gut can be 6-10 times that of the body. Inspired by this natural design, a known strain-engineering approach (referenced above) can be employed to create tortuous, air-channeling, filtration tubes by pre-stretching a piece of latex rubber sheet and bonding it onto the surface of a silicone rubber tube. Upon release of the stretched structure, the mismatch in the strain will result in the formation of a hemihelical structure 400 as shown in FIGS. 4 and 5. The stretched version of an exemplary filtration tube 600 is further depicted in FIG. 6. In an exemplary embodiment, the filtration tubes in each filtration assembly 230, are arranged three on each side of the hosting compartment (within the bottom 232, with their respective inlets (at a first end) directed upwardly toward the wearer's mouth and nose upward adjacent the middle of the mask. A gap 310 resides in the bottom 232 between each set of filtration tube inlets sufficient in width to maintain structural integrity of the component. The opposing, outward ends of each of the filtration tubes are, conversely, directed downwardly to communicate with ambient air in the environment external of the mask 200.

Recent studies show that SARS-CoV-2 mainly spread through respiratory droplets/aerosols produced when an infected person coughs, sneezes, or talks. According to recent data, the virus can remain viable/active for up to three days on plastic and steel, but once it lands on a surface, the amount of viable virus begins to disintegrate in a matter of hours. Hence, the structure of the mask is directed toward disabling the transmission capability of SARS-CoV-2 through the air by creating tortuous airway with unusually large surface areas so that the SARS-CoV-2 would no longer be able to travel through the tubes within their survival on the inner surface, in which case the embodiments herein serve to minimize the need for highly demanded melt-blown filter fabrics in this next-generation mask with superior breathability.

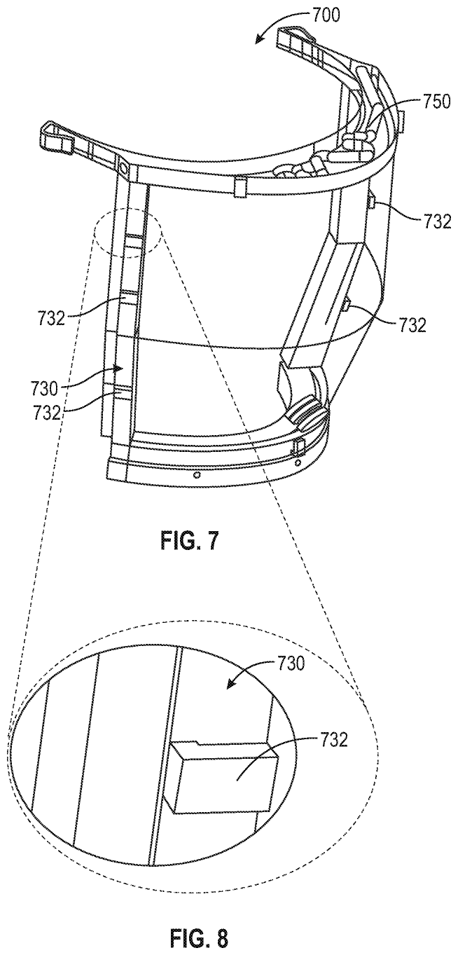

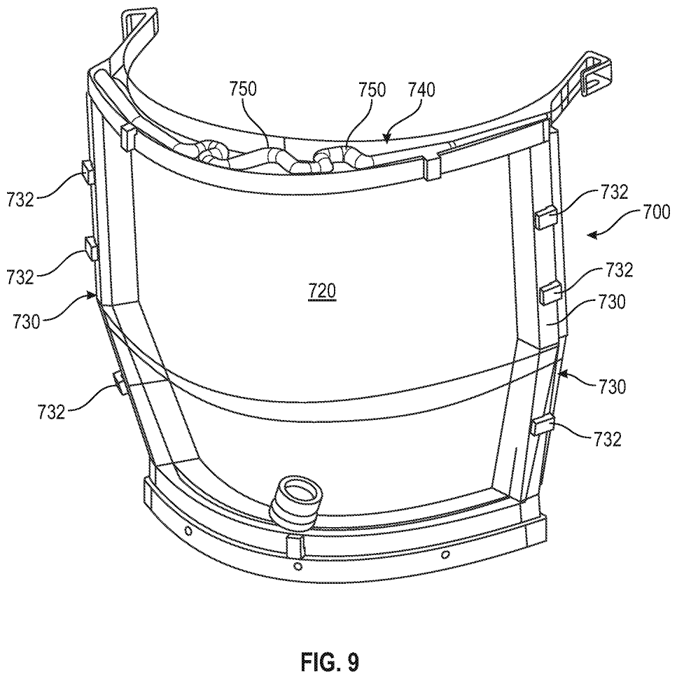

Another embodiment of the see-through full face mask 700 with tortuous filtration tubes is shown in FIGS. 7-9. The general construction of this embodiment is similar to the mask 160 described with reference to FIGS. 1A and 1B above. It includes a transparent rigid, or semi rigid (or deformable) visor 720 surrounded by a frame 730, which is secured by frame clips 732. Appropriate face seals are provided to the frame 730, as described above. In this embodiment, the mask 700 includes tortuous tube assemblies 750 built into a compartment in the head band 740, near the forehead. The headband compartment can be constructed as a unitary, molded structure, a 3D-pronted structure, a multicomponent assembly or a combination of such construction techniques. The shape and type of filtration tubes can be highly variable in accordance with the various designs described herein. In general, a sufficient number of such tubes should be provided to ensure free airflow and appropriate isolation of microdroplets. In an embodiment, the outward-facing inlets to each of the tubes in each assembly 750 are located on the outer edges of the headband 740, and the inner-facing inlets of the tubes are located near the center of the headband so as to direct flow toward the mouth and nose. Differing layouts and/orientations of inlets and outlets are contemplated in alternate embodiments.

### III. TORTUOUS AND CIRCUITOUS FILTRATION TUBE DESIGNS

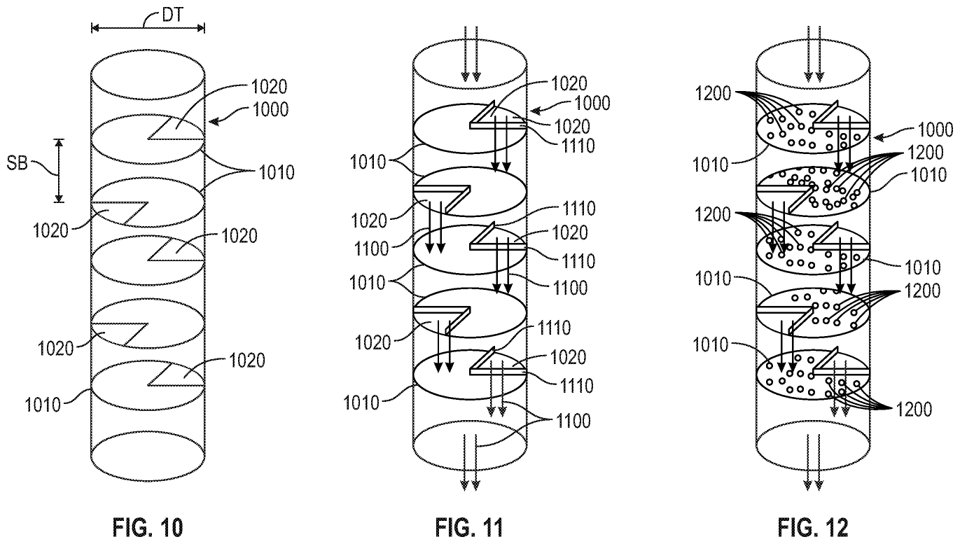

An alternate embodiment internal construction for a circuitous-path filtration tube 1000 is shown in FIGS. 10-12. This construction can be used in any of the mask embodiments described herein, and integrated in the bottom, top, frame and/or another portion of the mask. The depicted tube section 1000 is part of an overall elongated structure that can be straight (in a typical arrangement), or curved/serpentine, in shape. The tube can define any acceptable diameter DT for appropriate flow of air therethrough when ganged with a plurality of parallel tubes in a filtration tube assembly. The length of each overall tube is highly variable. For example, the diameter DT can be between a one and ten millimeters, while the overall tube length can be between 10 and 100 millimeters. The tube section 1000 is divided by spaced apart baffles 1010 that include (e.g.) pie-shaped openings 1020 of an appropriate arc size. Other shapes of openings are contemplated in alternate embodiments. The openings 1020 are arranged in an offset manner as shown so that they are out of alignment between adjacent baffles 1010.

As shown in FIG. 11, this results in an indirect flow (arrows 1100) through the tube 1000. As such, the microdroplets 1200 (FIG. 12) are captured on each descending baffle in the manner of a shelf. Hence, the transport of microdroplets is significantly hindered by the baffles 1010, and the time it takes for the microdroplets (and hence the virus carried therewith) to travel through the tube will become significantly longer—as long as it is longer than the time the virus can remain viable on the surface or the time needed for wearing the mask (which can then be disinfected for reuse). The spacing SB (FIG. 10) of baffles 1010 is highly variable—for example a few millimeters apart. The tube 1000 can be constructed in a variety of ways using commercially available production techniques—for example 3D printing, molding, and the like. Materials from which the tubes are constructed is also highly variable—for example conventional or anti-bacterial polymers and/or metals, such as copper—which has an anti-bacterial and anti-viral property that assists in making the filtration assembly essentially self-cleaning.

Optionally, each baffle can include a raised wall 1110 (FIG. 11) on either side edge of the baffle opening 1020, so as to further disrupt the flow of air and enhance the trapping of microdroplets. The opening walls 1110 are typically raised on the baffle side facing oncoming airflow, but can be raised on both sides where inhale and exhale air are passed through the same tube. In this context, it is contemplated that filtration assemblies can be made directional with conventional check valves on wither the inlet or outlet side (or both).

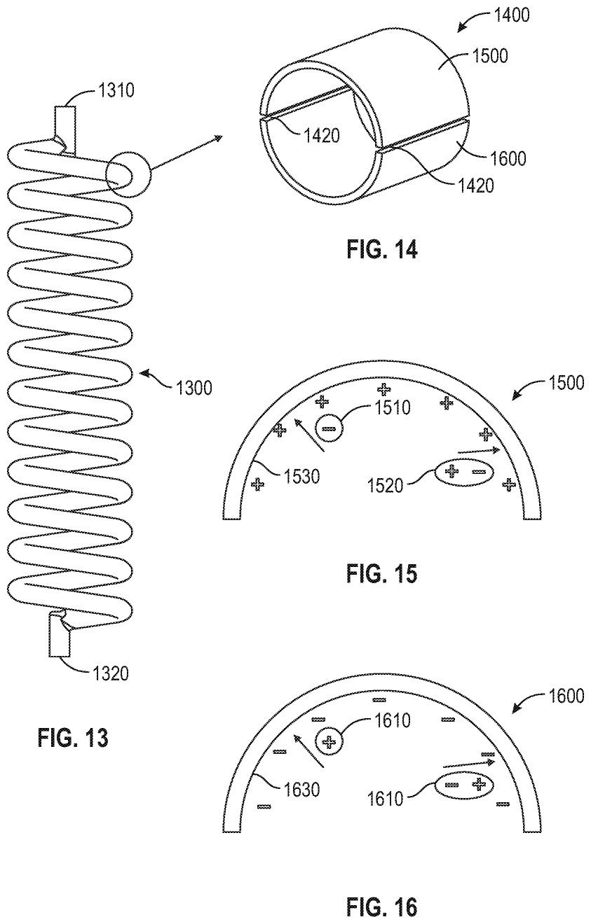

A spiral filtration tube 1300 with electrically charged attractive properties is shown in FIGS. 13-16. The tube 1300 is depicted as a spiral or helix with a plurality/multiplicity (in this example approximately 12) turns between an inlet 1310 and outlet 1320. The length and diameter of the tube is highly variable and, like other embodiments herein can be defined based upon the number of tubes in an assembly and the performance of the system. As shown in the cross section a s tube segment 1400 in FIG. 14, the tube defines an outer insulation layer and an inner charged layer—charge being provided by a disposable or rechargeable DC battery and/or or solar power source contained in the mask frame. The charged layer us divided (along gaps 1420) into a positive electrode 1500 (FIG. 15) and a negative electrode 1600 (FIG. 1600). The result of charging is that, as charged microdroplets 1510, 1610 pass through the tube 1300, they adhere to the oppositely-charged wall 1530, 1630. Likewise, neutral particles 1520, 1620 can pick up a charge/polarization via triboelectric charging as they pass down the charged tube, and thereby adhere to one of the walls.

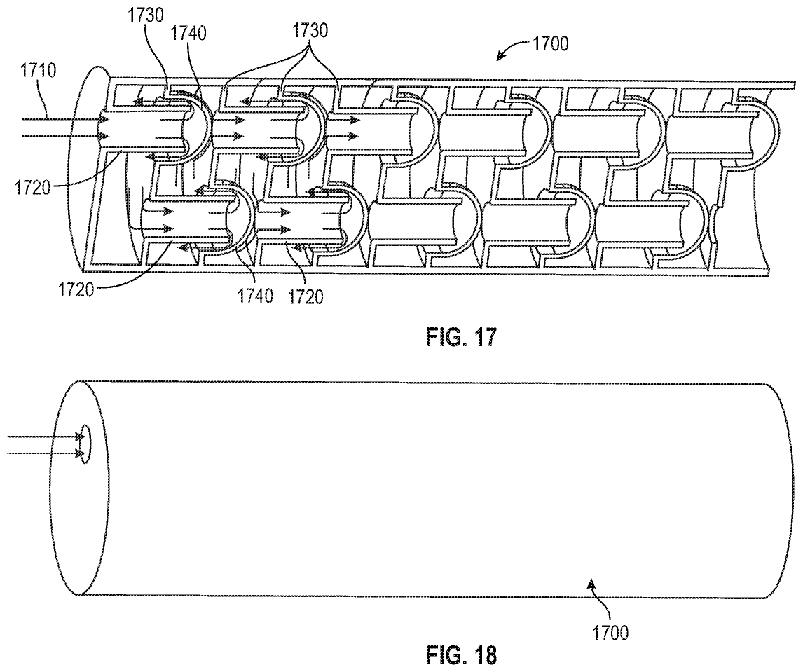

In yet another embodiment of a filtration tube 1700 shown in FIG. 17 (in cross section) and FIG. 18 (fully enclosed) a circuitous path of airflow (arrows 1710) is defined based upon entry into stem tubes 1720 that extend downstream 9in a flow direction) from walls 1730. The tubes are offset from each other and exit in a dome 1740 that allows passage from one chamber (between walls 1730) out of the stem tube and into the chamber of the next set of walls. This is one of a variety of geometries that can be defined by the interior of the tube 1700. The internal construction is generally designed to provide a highly indirect flow path with chambers that are sufficient in number and volume to capture the majority of microdroplets when air flows between inlet and outlet. As with other designs herein, bidirectional flow can occur with microdroplet capture in each direction thereby providing desirable filtration for both inhaled and exhaled air.

### IV. OPTIONAL MASK FEATURES

The above-described embodiments for a novel see-through full face mask enable a variety of desirable and beneficial features, including visibility, lightweight and comfortable fit, breathability and superior filtration capability through use of tortuous/circuitous-path filtration tubes. It is contemplated that these embodiments can be further enhanced with optional features that enhance functionality. Referring again to the embodiments of the mask 100 and 200 in FIGS. 1 and 2, respectively, the headband (or another portion of the frame, can include a vital-signs sensing array, including, for example, a conventional temperature sensor 190 (based on any acceptable mechanism—for example a thermocouple, IR, etc. The sensing assembly can include other types of sensors, such as a venous oximeter and/or pulse rate sensor, again implemented using conventional or custom technologies. Additionally, the mask 100, 200 can include a flow and/or cough/sneeze sensor (250 in FIG. 2). Such can operate e.g. by sensing differential pressure. These, sensors can communicate wireless via an controller module and (e.g.) WiFi or Bluetooth® wireless interface so as to provide the wearer (e.g. via a smartphone, tablet, etc.), and/or another interested party, such as a supervisor, with health information. Alternatively, a removable storage device, such as a USB-based memory stick or removable FLASH drive, can be used to store and download telemetry and program instructions

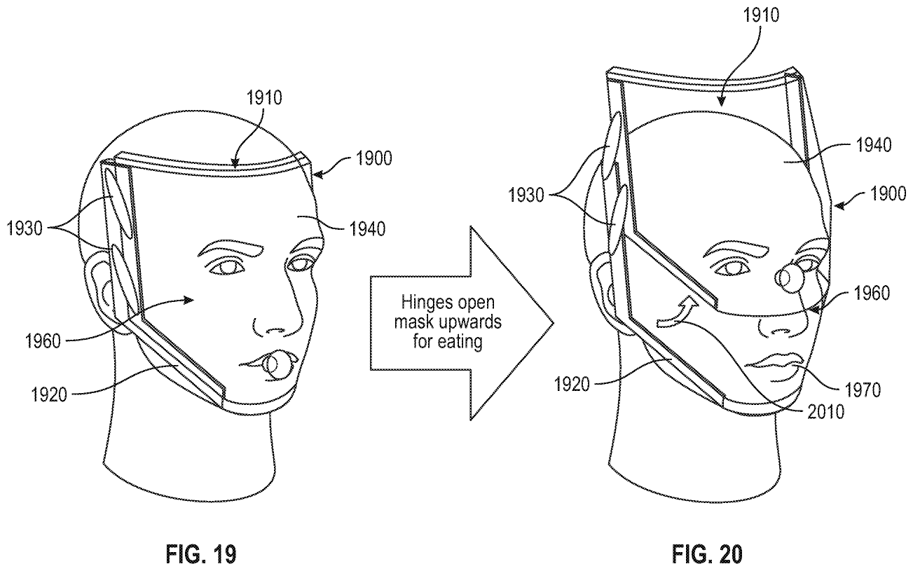

A significant disadvantage to wearing any type of face mask is the inability to eat or drink while it is applied. One of the top reasons for not wearing a face mask is that one cannot drink or eat with a face mask on. With reference to FIGS. 19-22, an embodiment of the mask 1900 generally described herein, which allows for both eating and drinking while the mask is still worn. The visor is part of a subframe assembly 1910 that is shown closed in FIG. 19 to provide protection. In this mode, the subframe (which can be constructed from a pliable or sealing material) surrounds the transparent (rigid or semi-rigid) visor 1940, and is secured against a base frame 1920 constructed from a rigid (e.g.) polymer material. The base frame 1920 seals against the wearer's face 1960 as shown using appropriate retaining bands or straps as described above, including head bands and neck bands, among others. The subframe 1910 is hinged via link bars 1930 as shown (curves arrow 2010) in FIG. 20 so as to allow the entire visor 1940 to be lifted away from the users mouth area 1970. This allows the user to eat and drink without (free of) full removal of the mask. Moreover, since the swinging action is quick and intuitive, it minimizes the length of time that the wearer must expose himself or herself to the environment. When finished, the wearer simply swings the subframe 1910 back into a sealed against the base frame 1920 as shown in FIG. 19. Note that the base frame 1920 can include appropriate filtration assemblies (fabric, tubes, etc.), in the headband or bottom as described above.

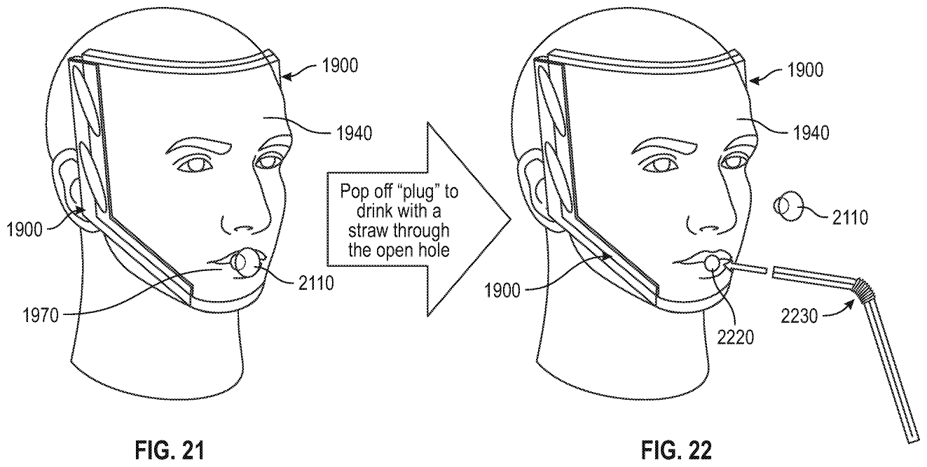

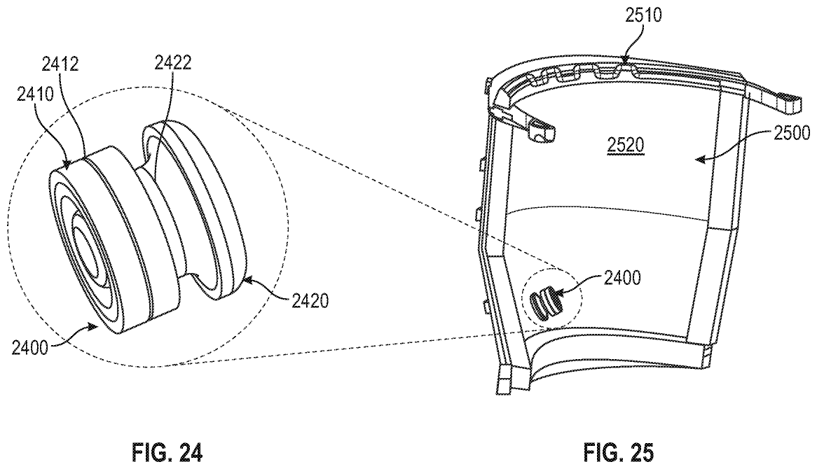

In FIGS. 21 and 22, the mask 1900 is shown in connection with operation of a drinking plug 2110, which is located in the bottom middle of the visor 1940 in a position that resides over the wearer's mouth area 1970. The drinking plug 2110 can be provided as part of the depicted swinging mask 1900, or as part of a fixed-position mask as described below. Referencing FIG. 22, the drinking plug 2110 normally seals an opening 2220 in the visor 1940, but can allow a straw 2230 to be inserted when removed, so that the wearer can draw fluid from a vessel with his or her mouth. A version of the plug assembly 2400 is shown in FIG. 24. It consists of a base grommet 2410 with a circumferential detent 2412 that is adapted to seat within a hole in the visor. The grommet 2400 includes a central hole that receives a stem 2422 of the plug 2420 in a friction fit. When the plug 2420 is removed from the grommet 2410, the hole in the grommet is sufficient in diameter to allow passage of a conventional straw. It can be slightly larger than the straw or allow for a slight frictional engagement to maintain an airtight seal while the straw is inserted. FIG. 25 shows a rear view of an exemplary mask 2500 according to an embodiment with a filtration assembly mounted in the headband 2510 and the drinking plug 2400 attached to the visor 2520.

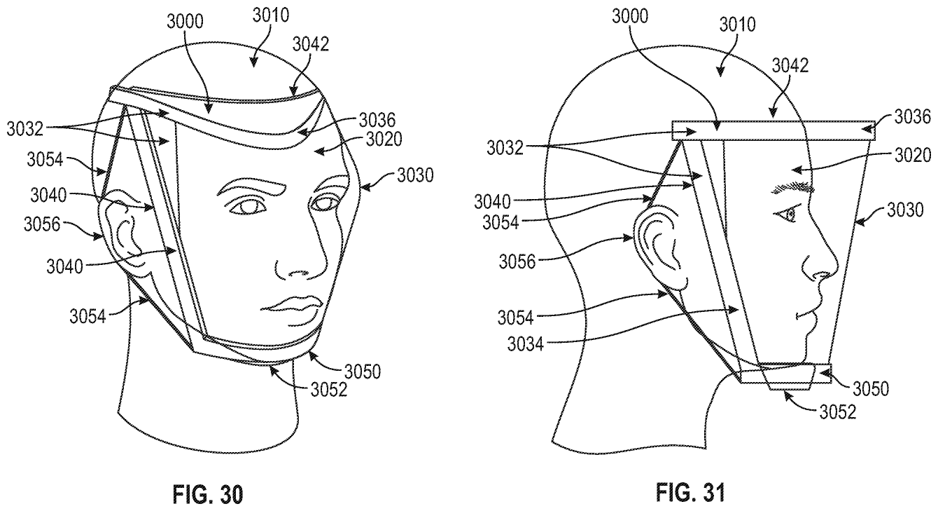

In FIGS. 30 and 31, the see-through full face mask 3000 is shown applied to a wearer's head 3010 and face 3020. Similar in structure and function to the previously described face mask 100, this mask 3000 surrounds the transparent visor 3030 (e.g. rigid/semi-rigid) with a polymer frame 3032. The vertically oriented sides 3034 and top headband 3036 of the frame 3032 are oriented so as to conform to the general dimensions of an average face of a given size, and the frame sides 3034 and headband 3036 can, respectively, include strips 3040 and 3042 of a compliant material, such a silicone and/or foam, that conforms to the face to provide a relatively airtight seal between the mask and wearer's face, and also provides enhanced comfort to the wearer. Secured to the mask's base 3050 is an antiviral and/or particulate filter 3052 to further hinder pathogen-carrying microdroplets from entering into and escaping the mask 3000. The frame 3032 is biased against the wearer's face by elastic straps 3054 to be wrapped around the ears 3056. Looping around each ear 3056, the straps 3054 are attached adjacent to top and bottom corners of the frame sides 3034. The depicted strap assembly involves adjustable ear loops, but the strap assembly can include adjustment slots, buckles or a hook-and-loop closure to vary the length thereof for differing sized heads.

Note that the dimensions shown in FIG. 25, FIG. 30, and other illustrations herein are exemplary of a variety of possible arrangements that can vary, in part based upon the size of the wearer's face—for example, masks can be provided in a range of sizes, such as small, medium, large and extra-large. A wearer is fitted for the appropriate size using a sizing procedure that should be clear to those of skill. Alternatively, certain sealing arrangements can provide for a universal fit within a given range of face sizes.

In various embodiments, the above-described tortuous or circuitous-path filtration tubes can be used to regulate temperature inside the mask. This can be accomplished manually by the user or automatically using (e.g.) the circuitry embedded in the headband or bottom as described generally in FIGS. 1 and 2 above. Where performed automatically, an internal temperature probe measures the temperature within the airspace of the mask and adjusts appropriate components of the mask. For example, to make the wearer feel cooler in the summer or when exercising, and warmer in the winter or when the ambient air is cold, the exterior and/or interior cross sections of the respiratory tubes can be different sizes. When the interior cross section is smaller than the exterior, the tube defines a nozzle, so the air inhaled will be cooler than the outside. Conversely, when the interior cross section is larger than the exterior, the tube becomes a diffuser, so that the air inhaled is warmer than the outside. Since there are typically multiple tubes ganged together in a single integrated filtration assembly, the inlet or outlet of the assembly can be a single port and incorporate an adjustable/removable cap, similar to the drinking plug of FIG. 7—or an automatic, electrically actuated valve operatively connected to a temperature regulation system in the circuitry described above. The cap or valve thereby allows the degree of airflow to be changed to regulate temperature.

### V. FACE SHIELDING HOODS

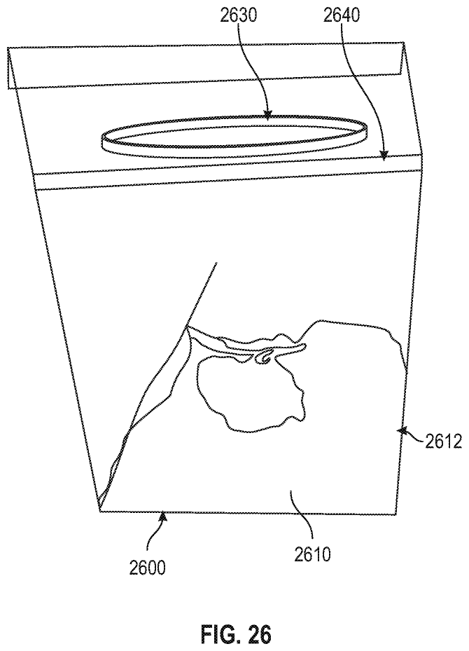

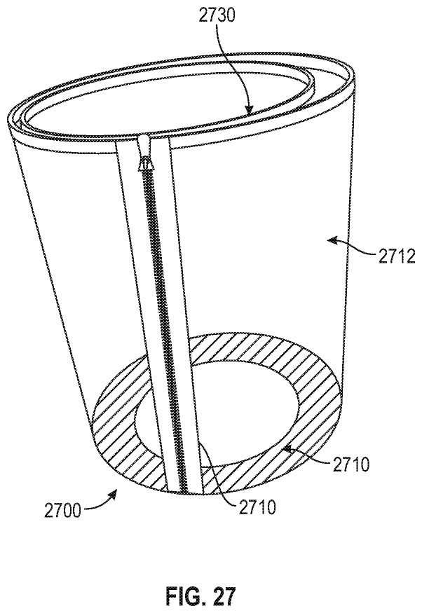

In an alternate embodiment, a see-through full face-shielding hood is described. Such can be constructed using a variety of techniques, including molding, extruding and 3D printing. Hoods are characterized by full coverage of the head region with associated filtration in the bottom or headband. Hence such hoods enclose the entire head of the wearer and can be see-through from all sides. Referring to FIGS. 26 and 27, two respective hood designs 2600 and 2700 are shown. The hood bottom (2610 and 2710, respectively) is enclosed with soft fabrics that can be stretchable and breathable, or can be adapted seal so as to allow air to pass through a filtration tube assembly as described above. In the embodiment 2600 of FIG. 26, the fabric 2610 near the neck is deformable, so it is easy to put it on and off. In the embodiment 2700 of FIG. 27, the bottom 2710 fits snugly to the neck. Each hood 2600, 2700, respectively, comprises a thin, pliable transparent polymer shield 2612, 2712, similar in sheet material to the deformable visor described above. To accommodate the closer/snugger bottom 2710, the hood 2700 includes a side zipper 2720 to allow the hood to be more conveniently put on and off. Each hood 2600 and 2700 includes a headband 2630. 2730, respectively with appropriate cushioning and/or seal to engage the crown of the wearer's head. The hood 2600 defines a cross section that is generally square and is supported at its top by an outer frame 2640 that connects with the headband 2630. The area between the outer frame 2640 and the headband can be filled with an appropriate material—for example sealed or breathable cloth, as appropriate.

### V. HALF SEE-THROUGH FACE MASKS

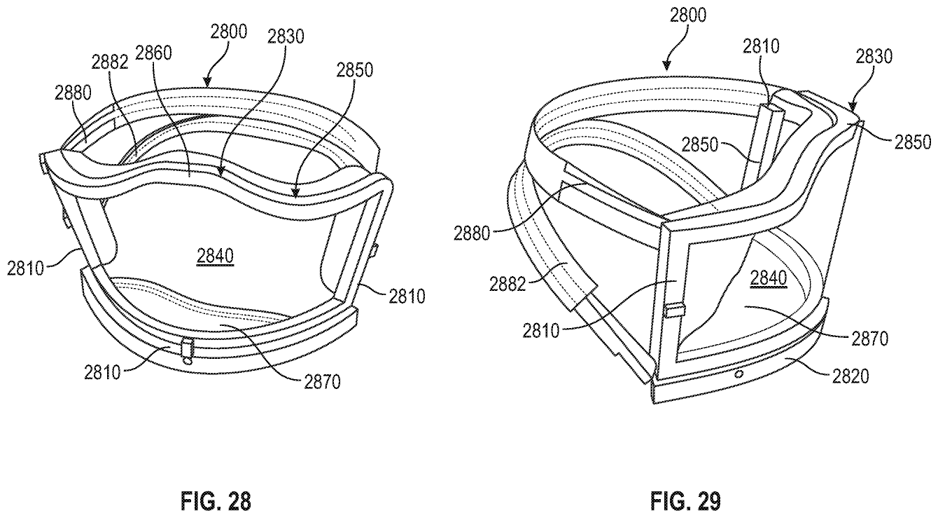

Reference is made to FIGS. 28 and 29 that depict a “half” face mask 2800 according to an exemplary embodiment. This embodiment can be deistable where visibility and superior filtration are advantageous but a full face mask or hood is not convenient or desired. The half face mask 2800 defines a polymer frame consisting of sides, 2810, bottom 2820 and top 2830. The frame can be semi-rigid, or typically, flexible to conform to a range of facial sizes and shapes. For example, the frame can be constructed of a molded silicone or rubber. The frame supports a clear/transparent “visor” or face plate 2840, typically constructed from a deformable polymer sheet so as to conform, with the frame to the wearer's face. The frame includes a sealing foam (and/or other pliable material, such as silicone) surround 2850 that cushions the fit for comfort. The top 2830 of the mask includes an upward contour 2860 to accommodate the wearer's nose. For the purposes of the description, this element can be considered the headband in certain implementations—that is for the purpose of integrating filtration tube assemblies. More particularly, the frame bottom 2820 of the depicted embodiment defines a standoff from the wearer's chin that is filled with a fabric structure 2870 (one or more layers of fabric/filter material) that provides a filtration assembly. This material structure 2870 impinges against the chin so create an approximate seal. In alternate embodiments, the frame bottom can include a filtration tube assembly, according to the various embodiments described above, with appropriate sealing structures against the wearer's chin.

The frame is biased against the wearer's face by a pair of straps 2880 and 2882 that are generally elastic (e.g. elastic webbings) and pass around the wearer's head and/or neck. The straps 2880 and 2882 are attached adjacent to top and bottom corners of the frame sides 2810. The strap assembly can include adjustment slots, buckles or a hook-and-loop closure to vary the length thereof for differing sized heads. Other retaining mechanisms can be employed—for example adjustable ear loops or a cap system.

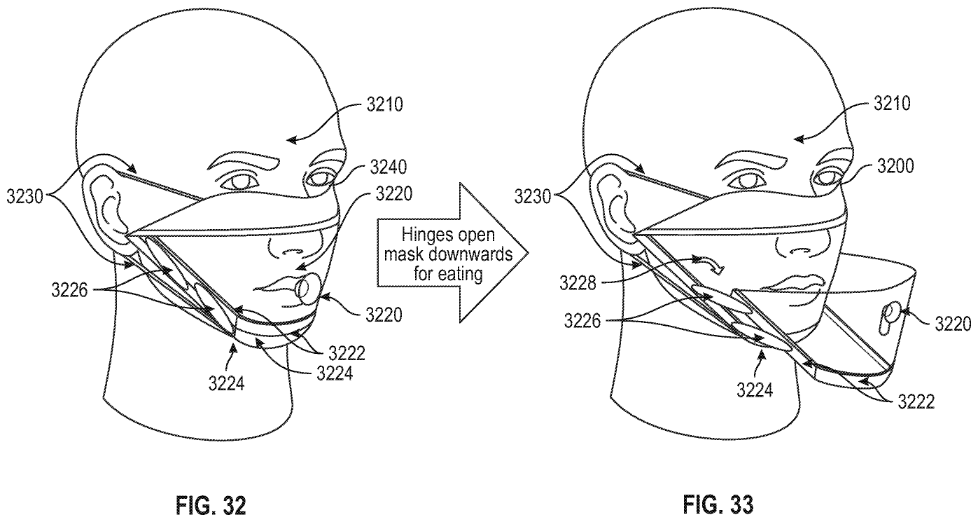

In FIGS. 32-35, a see-through half face mask 3200 is shown applied to a wearer's face 3210. Similar to the see-through full face mask 1900 depicted in FIGS. 19-22, this half face mask 3200 allows for both eating and drinking while the mask is still worn. The transparent visor 3220 is part of a subframe assembly 3222 that is shown closed in FIG. 23 to provide protection. In this mode, the subframe assembly 3222 is secured against a base frame 3224 constructed from a rigid polymer material. The base frame 3224 seals against the wearer's face 3210 as shown using appropriate retaining bands or straps 3230 at the ears, although the strap assembly may include head bands and neck bands, among others. The subframe 3224 is hinged via link bars 3226 as shown (curved arrow 3228) in FIG. 33 so as to allow the entire visor 3220 to be lifted away from the user's mouth area 3240, allowing the user to eat and drink without full removal of the mask. When finished, the wearer simply swings the subframe 3222 back into a sealed position against the base frame 3224 as shown in FIG. 32.

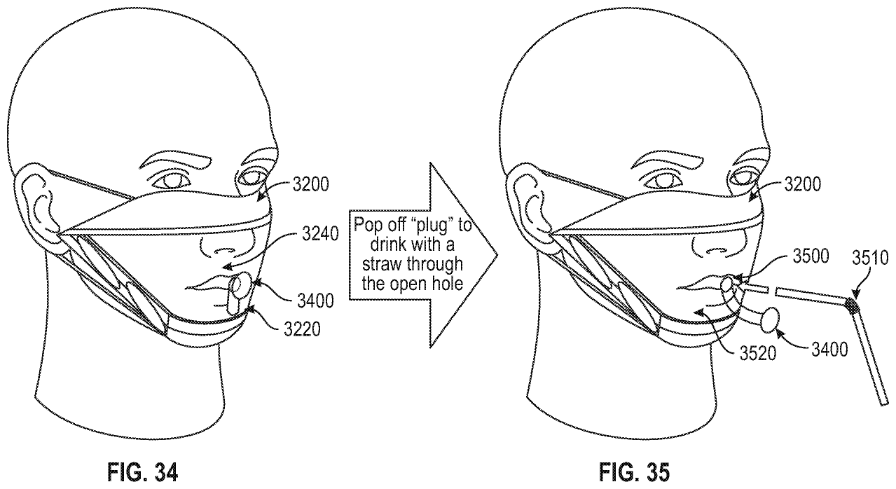

In FIGS. 34 and 35, the mask 3200 is shown in connection with operation of a drinking plug 3400, which is located in the middle of the visor 3220 in a position that resides over the wearer's mouth area 3240. Referencing FIG. 34, the drinking plug 3400 normally seals an opening 3500 in the visor 3220 but can allow a straw 3510 to be inserted when removed, so that the wearer can draw fluid from a vessel with his or her mouth. The plug assembly, shown in FIG. 35, consists of a grommet at the visor's opening 3500, connected to a flexible strap 3520 that holds the drinking plug 3400 at its end. Having the plug assembly as one joined piece at the visor serves to prevent the small drinking plug 3400 from becoming separated from the mask 3200.

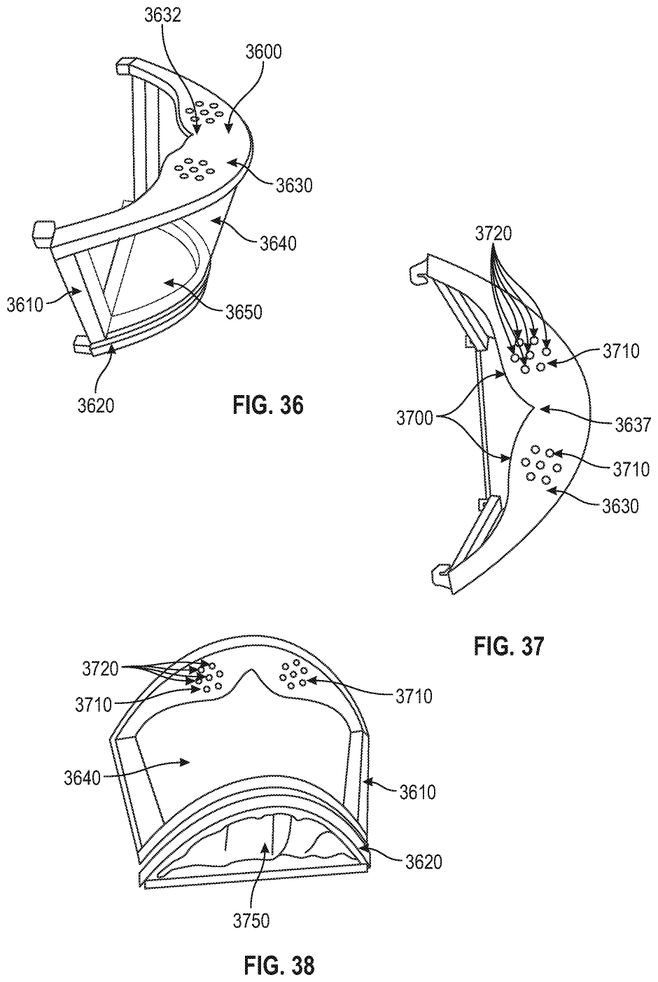

Reference is made to FIGS. 36-38 that depict a see-through half face mask 3600, similar to the half mask depicted in FIGS. 28 and 29. The half face mask 3600 defines a rigid, semi-rigid, or flexible polymer frame consisting of sides, 3610, bottom 3620 and top 3630. Conforming to the wearer's face, the frame also supports a clear/transparent visor 3640. The top 3630 of the mask includes a flexible upward contour 3632 to accommodate the wearer's nose. The frame bottom 3620 of the depicted embodiment defines a standoff from the wearer's chin that is filled with a fabric structure 3650 (one or more layers of fabric/filter material) that provides a filtration assembly. This material structure 3650 impinges against the chin to create an approximate seal.

Shown in FIG. 37, the top 3630 of the mask 3600 includes two slots 3700 to insert a small, flat, square-shaped aluminum sheet piece 3710 into each slot opening. When aluminum sheet pieces 3710 are not inserted, the slots 3700 are exposed to the outside environment via holes 3720. When aluminum sheet pieces 3710 are inserted, the aluminum sheet pieces 3710 serve to amplify the sound of the user's speech by carrying sound waves from within the mask to outside of the mask, allowing surrounding listeners to hear the user's speech at greater volumes. Aluminum sheet is a material that conducts sound well, and the insertion of aluminum sheet pieces 3710 into the mask 3600 serves to amplify sound through the holes 3720 at the areas where the slots 3700 reside.

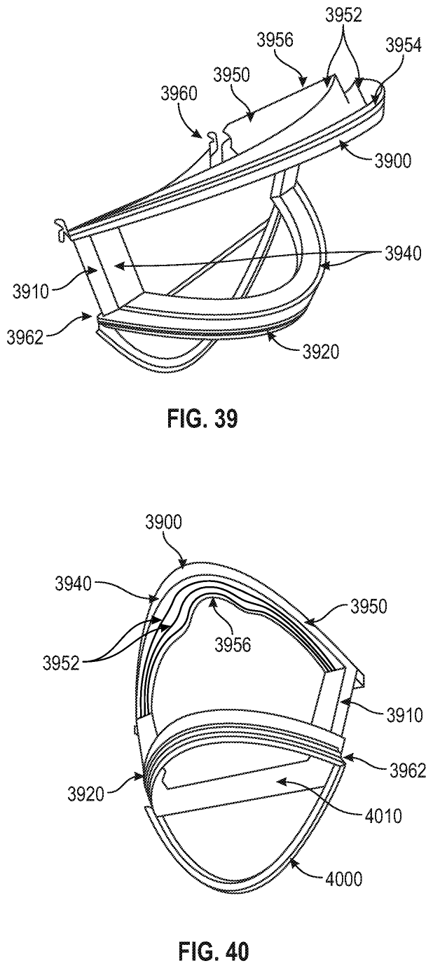

In FIGS. 39-42, a naked half face mask frame 3900 is shown, with neither transparent visor nor filter present. Lining the perimeter of the sides 3910, bottom 3920, and top 3950 of the mask frame is a continuous cleft or indentation 3940. Serving the same purpose as the set of clips 176 depicted in FIGS. 1A-1B, the indentation 3940 secures a transparent conically folded visor into the frame 3900. Running continuously along the mask frame, the indentation 3940 ensures that the environment inside the mask is sufficiently separated from the environment outside of the mask. The top 3950 of the mask frame incorporates continuous accordion-shaped ridges 3952 stretching from the outside perimeter of the top of the frame 3954 to the inner upward contour 3956 that accommodates the wearer's nose. The accordion-shaped ridges 3952 serve to make the upward contour area 3956 touching the nose and cheeks more flexible, allowing for a more comfortable and universal fit at the nose and cheek area within a given range of face sizes. Top hooks 3960 at the frame top 3950 and bottom hooks 3962 at the frame bottom 3920 serve as areas of attachment for elastic bands or straps, to be wrapped around the head, neck, or ears for secure attachment of the mask to the face.

In FIGS. 40-42, the bottom 3920 of the frame includes an overhanging clamp mechanism 4000 in open formation. The clamp mechanism 4000 serves to hold a semi-ovular piece of filter (e.g., polypropylene, one or more layers of fabric, among others). When the piece of filter is inserted, the clamp mechanism 4000 can be moved to the bottom 3920 of the frame and snapped into place via the clamp's flexible hinge 4010, converting the clamp mechanism from its open formation into its closed formation, keeping the piece of filter secured into place. While the clamp mechanism 4000 depicted is semi-ovular in shape, the entire clamp assembly, including the overhang, frame bottom 3920, and hinge 4010, can take the form of any theoretical shape that still allows fixture of a piece of filter at the bottom of the mask frame.

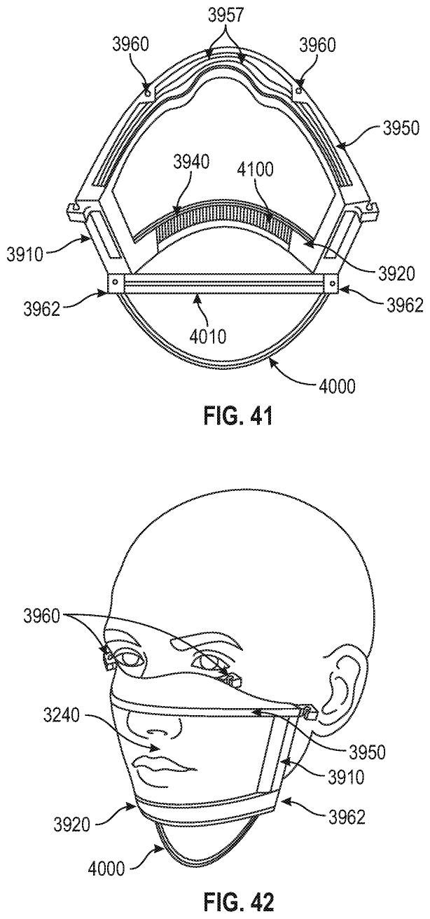

In FIG. 41, the rear of the bottom 3920 of the frame is serrated in accordion-shaped notches 4100, serving to make the bottom 3920 of the mask more flexible, allowing for a more comfortable and universal fit at the lower cheek area within a given range of face sizes.

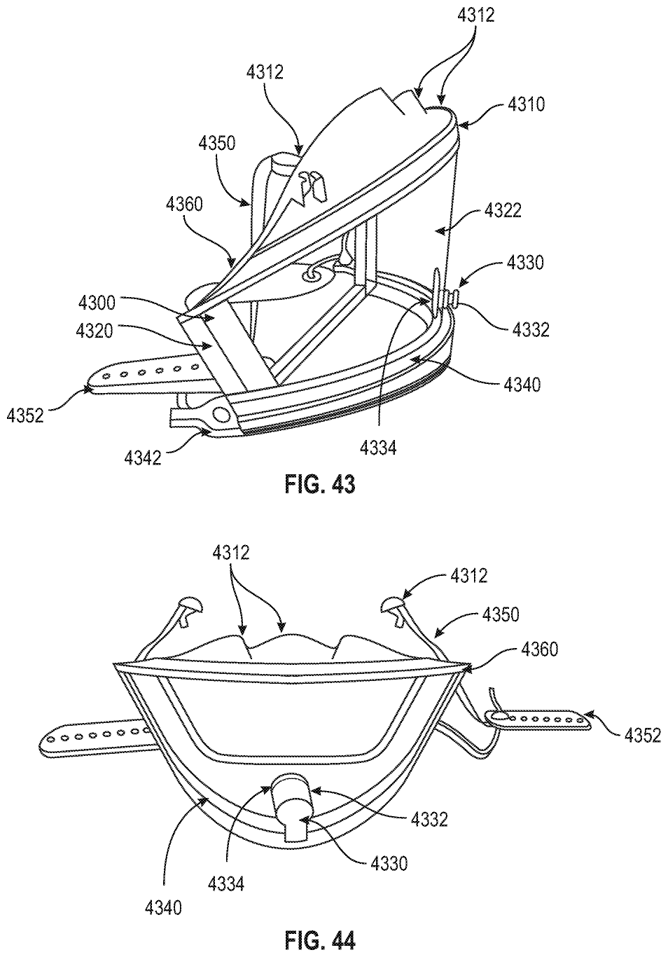

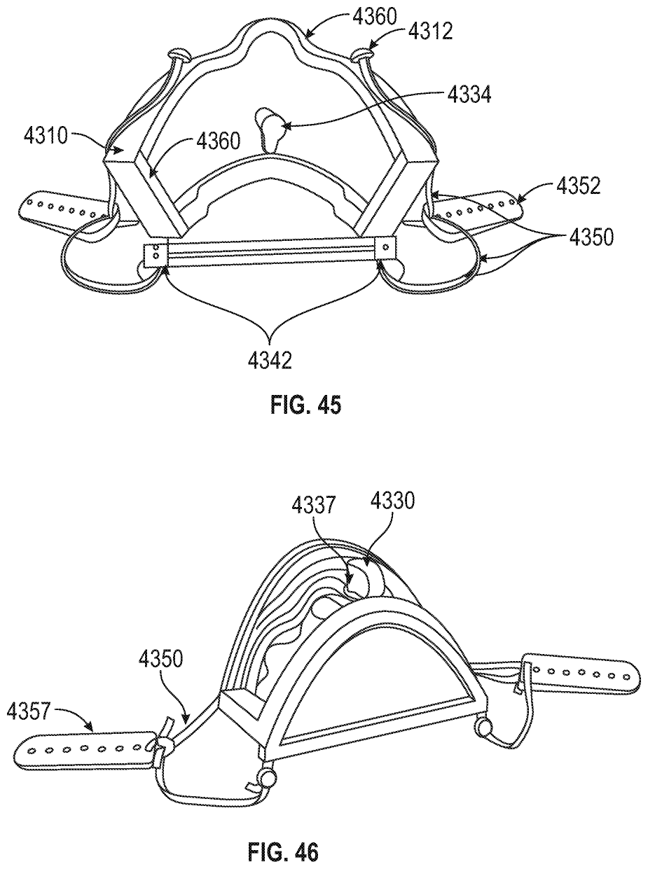

Reference is made to FIGS. 43-48 that depict a see-through half face mask 4300, similar to the half mask frame depicted in FIGS. 39-42. The top 4310 of the mask includes accordion-shaped ridges 4312 for flexibility and face fitting. The polymer frame of the mask 4320 holds a transparent visor 4322. On the transparent visor is a drinking plug assembly 4330 for straw insertion purposes. The drinking plug assembly 4330 includes a grommet 4332 attached at the lower center of the visor 4322, with a rubber ring 4334 at the rear of the visor to secure the grommet 4332 in place at the visor 4322. Top hooks 4312 at the frame top 4310 and bottom hooks 4342 at the frame bottom 4340 serve as areas of attachment for elastic bands or straps 4350, to be wrapped around the back of the head. Located at the ends of each elastic strap is an adjustment buckle 4352, allowing varying fit for different head sizes. The depicted strap assembly involves head straps 4350 and an adjustment buckle 4352, but the strap assembly can include adjustment slots, hook-and-loop closures, and other mechanisms to vary the length thereof for differing sized heads. The mask frame 4320 include strips 4360 of a compliant material, such a silicone and/or foam, that conforms to the face to provide a relatively airtight seal between the mask and wearer's face, and also provides enhanced comfort to the wearer.

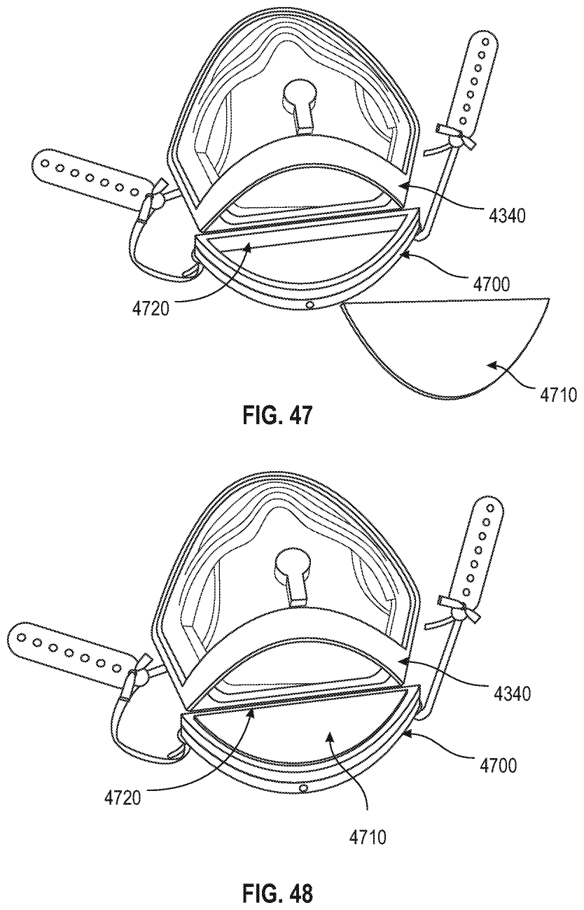

In FIGS. 47 and 48, the mask 4300 is shown in connection with operation of a filter clamp mechanism 4700, which is located at the bottom 4340 of the mask. The filter clamp 4700 in FIG. 47 serves to hold a semi-ovular piece of filter 4710 (e.g., polypropylene, one or more layers of fabric, among others). The filter clamp 4700 in FIG. 47 is depicted in its open formation. When the piece of filter 4710 is inserted, the clamp mechanism 4700 can be moved to the bottom 4340 of the frame and snapped into place via the clamp's flexible hinge 4720, converting the clamp mechanism from its open formation into its closed formation, keeping the piece of filter secured into place. While the clamp mechanism 4700 depicted is semi-ovular in shape, the entire clamp assembly, including the overhang, frame bottom 4340, and hinge 4720, can take the form of any theoretical shape that still allows fixture of a piece of filter at the bottom of the mask frame.

### VI. CONCLUSION

It could be clear that the see-through full and half face mask and hood embodiments herein provide a superior form of protection against airborne viruses and other contaminants without sacrificing comfort or visibility. More generally, they are lightweight, clear, comfortable for long-term wear, with superior breathability, reusable, and effective in preventing the transmission through respiratory microdroplets and aerosols. These embodiments can serve to upgrade our health infrastructure and ecosystems, dramatically enhancing preparedness for epidemics and pandemics and to further protect health workers, caregivers, first responders, vulnerable people and the public. These face shield and hoods can be reused with relatively straightforward cleaning techniques. This reduces waste and potential cross contamination via disposal of PPE. The embodiments herein can be mass-produced using conventional techniques, and can potentially replace traditional facemasks and face shields in many applications.

The foregoing has been a detailed description of illustrative embodiments of the invention. Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments of the apparatus and method of the present invention, what has been described herein is merely illustrative of the application of the principles of the present invention. For example, as used herein, the terms “process” and/or “processor” should be taken broadly to include a variety of electronic hardware and/or software based functions and components (and can alternatively be termed functional “modules” or “elements”). Moreover, a depicted process or processor can be combined with other processes and/or processors or divided into various sub-processes or sub-processors. Such sub-processes and/or sub-processors can be variously combined according to embodiments herein. Likewise, it is expressly contemplated that any function, process and/or processor herein can be implemented using electronic hardware, software consisting of a non-transitory computer-readable medium of program instructions, or a combination of hardware and software. Additionally, as used herein various directional and dispositional terms such as “vertical”, “horizontal”, “up”, “down”, “bottom”, “top”, “side”, “front”, “rear”, “left”, “right”, and the like, are used only as relative conventions and not as absolute directions/dispositions with respect to a fixed coordinate space, such as the acting direction of gravity. Additionally, where the term “substantially” or “approximately” is employed with respect to a given measurement, value or characteristic, it refers to a quantity that is within a normal operating range to achieve desired results, but that includes some variability due to inherent inaccuracy and error within the allowed tolerances of the system (e.g., 1-5 percent). Accordingly, this description is meant to be taken only by way of example, and not to otherwise limit the scope of this invention.

What is claimed is:

1. A see-through face mask comprising:
a frame having at least one of a top, bottom and sides, that engages a face of a wearer and provides a cushioned approximate seal thereagainst;
a strap system that maintains the frame against a face of the wearer;
a visor that is substantially transparent and is supported by the frame; and
a filtration assembly that resides in the frame and that allows passage of inhaled and exhaled air therethrough.

2. The see-through face mask as set forth in claim 1 wherein the filtration assembly comprises a plurality of tortuous filtration tubes that are constructed and arranged to trap microdroplets in the air as it passes therethrough.

3. The see-through face mask as set forth in claim 1 wherein the tortuous filtration tubes each comprise a strain-engineered elastomer structure.

4. The see-through face mask as set forth in claim 1 wherein the tortuous filtration tubes comprise a plurality of internal baffles with openings in an offset arrangement therebetween.

5. The see-through face mask as set forth in claim 4 wherein the internal baffles include stem tubes and diverting domes.

6. The see-through face mask as set forth in claim 1 wherein the frame and the visor defines a full face mask and the filtration assembly is located in at least one of the top and the bottom of the frame.

7. The see-through face mask as set forth in claim 6 wherein the frame and the visor define a half face mask and the filtration assembly is located in at least one of the top and the bottom of the frame.

8. The see-through face mask as set forth in claim 1 wherein the frame is adapted to remain attached to a wearer's face while a subframe surrounding the visor is movable away from the frame to allow access to a mouth of the wearer.

9. The see-through face mask as set forth in claim 1 wherein the visor includes a removable drinking plug that allows insertion of a straw adjacent to a mouth of the wearer.

10. The see-through face mask as set forth in claim 1 wherein the frame is adapted to allow insertion of an antiviral and/or particular filter.

11. The see-through face mask as set forth in claim 1 wherein the frame is adapted to allow insertion of aluminum sheet pieces to amplify sound conduction of user speech.

12. The see-through face mask as set forth in claim 1 wherein the frame is adapted to allow incorporation of accordion-shaped ridges in the frame to increase flexibility, comfortability, and fit to different face shapes and sizes.

13. The see-through face mask as set forth in claim 1 wherein the frame is adapted to allow incorporation of a clamp mechanism to hold insertable pieces of filter.

14. A filtration assembly for a face mask comprising a plurality of tortuous filtration tubes that are constructed and arranged to trap microdroplets in the air as it passes therethrough.

15. The filtration assembly as set forth in claim 14 wherein the tortuous filtration tubes each comprise a strain-engineered elastomer structure.

16. The filtration assembly as set forth in claim 15 wherein the tortuous filtration tubes comprise a plurality of internal baffles with openings in an offset arrangement therebetween.

17. The filtration assembly as set forth in claim 15 wherein the internal baffles include stem tubes and diverting domes.

18. The filtration assembly as set forth in claim 17 wherein the face mask comprises either a full face mask or a half face mask.

19. The filtration assembly as set forth in claim 18 wherein the filtration assembly is located in at least one of a top and a bottom of a frame of the face mask.
